(19) United States
(12) Patent Application Publication
(10) Pub. No.: US 2014/0322085 A1
(43) Pub. Date: Oct. 30, 2014

(54) ODOR REMOVAL APPARATUS

(71) Applicant: LASKO HOLDINGS, INC., Wilmington, DE (US)

(72) Inventors: Joshua T. Hoffman, West Chester, PA (US); Philippe Genereux, Ottawa (CA)

(73) Assignee: LASKO HOLDINGS, INC., Wilmington, DE (US)

(21) Appl. No.: 14/261,955

(22) Filed: Apr. 25, 2014

Related U.S. Application Data
(60) Provisional application No. 61/817,461, filed on Apr. 30, 2013.

(51) Int. Cl. A61L 9/12

(52) U.S. Cl. CPC A61L 9/12
USPC 422/119; 422/123

(57) ABSTRACT

A portable odor removal apparatus for use in an enclosed space, such as for example a refrigerator and/or a closet. The portable odor removal apparatus includes; a housing, air inlets, air outlets, an ion generator and a controller with energy saving features. The overall form of the housing may suggest the appearance of food such as for example a fruit or vegetable. The device may also include hooks and other mounting apparatus.

### RELATED APPLICATION DATA

This application claims priority to U.S. provisional patent application Ser. No. 61/817,461 filed Apr. 30, 2013.

### FIELD OF THE INVENTION

The present invention relates to odor removal apparatus. More specifically, the present invention relates to odor removal apparatus having improved odor removal and performance characteristics for use within a confined space.

### BACKGROUND OF RELATED TECHNOLOGY

Enclosed spaces such as refrigerators, closets, and the like have long been known to develop odors and a general staleness of air. These odors are not desired in these enclosed spaces since they can migrate into items stored within the confined space.

For example, if the confined space is a refrigerator the items inside can generate odors. Items such as, onions, fish as well as other items undergoing natural decomposition have proven to be a source of unwanted odors. These odors entrapped in the enclosed space cause other items in refrigerator to absorb the odors and tastes.

Another example is a clothes closet. The majority of the items stored in such an enclosed space are mainly clothing. Also other items apart from clothing might be stored in a closet; such object may carry unwanted odors. The lack of ventilation in a closet increases the development of stale air and entraps any unwanted odors and smells within.

Several conventional responses have been used to remove unwanted odors in enclosed spaces. Some devices use materials such as baking soda or charcoal that have an ability to absorb odors. A disadvantage of these devices is the need to replace the materials periodically as maximum effectiveness is reduced over time. The necessity to replace the materials is usually not discovered until odors once again begin to be detected in the enclosed space. The presence of odors defeats the initial purpose of these devices and places the items within the enclosed space at risks of odor contamination.

Other conventional devices have been developed that do not remove odors but add additional fragrances and smells to an enclosed space to essentially cover up and/or mask the unwanted odors. These devices are not useful if the enclosed space is a refrigerator since the added odor would only serve to taint the taste of the other items in the refrigerator. Devices that mask or attempt to cover-up unwanted odors have only limited use if the enclosed space is a closet. Such additional odors over time accumulate in clothing items, causing all of the items within the closet to have a particular odor. Also most individuals do not desire an additional odor but would prefer no additional odor.

Other disadvantages associated with conventional devices are stability and aesthetic. The vertical aspect ratio of the conventional device is inherently unstable and can easily be over-turned with minimal impact. The vertical aspect ratio of the structure of conventional devices does not offer the opportunity of a design form and a shape that would be more pleasing and applicable in specific applications.

In short these conventional devices fail to meet the end users' needs in an adequate function and/or fashion. The end user needs to constantly monitor the condition of the device to know when to replenish odor absorption materials. Devices that add “cover-up” odors and smells have a very limited use and appeal. The cost to purchase and maintain some of these devices can be a burden.

### SUMMARY OF THE INVENTION

In view of the deficiencies of the prior art the following is a description of an odor removal apparatus with improved odor removal and performance characteristics.

As described herein, an odor removal apparatus according to the present invention utilizes an innovative structure and natural molecular reactions to remove—not cover up—odors in an enclosed space. The ability of the present invention to monitor itself and alert the end user to required maintenance is also an advantage that results in consistent odor removal.

Another advantage of the present invention includes a minimal use of electric power and a naturally induced air movement through the device. This reduces the cost and frequency of maintenance. Yet another advantage of the present invention is directed toward the construction of the device which is simple. This construction lowers the cost of manufacturing and subsequently to the end user.

The present invention uses distributed ozone to naturally eliminate odors. Ozone is one of the most effective ways to destroy and eliminate odors in lieu of simply masking or covering the odors. The innovative structure of the current invention produces an effective quantity of ozone while assuring that the production of ozone complies with industrial and statutory requirements.

In short, the ability of the present invention to use one of the most effective and natural odor removal processes combined with low energy usage and the ability of the apparatus to alert the end user to maintenance needs is both a needed and a desired innovation.

In certain exemplary, non-limiting embodiments, the present inventive is directed to a portable odor removal apparatus, the portable odor removal apparatus including: a housing wall defining an interior space; an ion generator located in the interior space and including an emitter electrode and an attractor electrode; a voltage source capable of providing voltage to the ion generator; an air inlet in the housing wall; at least one non-conductive diffusion surface located above the ion generator; and an exit passageway at least partially located in the non-conductive surface; wherein, voltage provided to the ion generator from the voltage source induces an electrical bias differential between the emitter electrode and the attractor electrode; the electrical bias differential creating an ion field having a flow of ions from the emitter electrode toward the attractor electrode resulting in the production of ozone; and the flow of ions entraining air molecules to cause air outside the housing wall to enter the interior space through the air inlet and mix with the ozone to produce ozonized air; and wherein the ozonized air exits the interior space through the exit passageway.

In certain exemplary, non-limiting embodiments, the attractor electrode defines a flow through area.

In certain exemplary, non-limiting embodiments, the exit passageway includes multiple direct exit passageways defining a total direct exit area which is equal to or less than the flow through area defined by the attractor electrode.

In certain exemplary, non-limiting embodiments, the exit passageway includes multiple indirect exit passageways defining a total indirect exit area which is equal to or greater than the flow through area defined by the attractor electrode.

In certain exemplary, non-limiting embodiments, the air inlet includes multiple air inlets defining a total inlet area which is equal to or greater than the flow through area defined by the attractor electrode.

In certain exemplary, non-limiting embodiments, the portable odor removal apparatus further includes a controller having a timer capable of cycling the electrical bias differential on and off at predetermined time intervals.

In certain exemplary, non-limiting embodiments, the electrical bias differential results from a negative charge on the emitter electrode and a grounding of the attractor electrode.

In certain exemplary, non-limiting embodiments, the electrical bias differential results from a first charge having a first polarity on the emitter electrode and a second charge having a second polarity on the attractor electrode, wherein the first and the second polarities are opposite.

In certain exemplary, non-limiting embodiments, the voltage source includes at least one battery located within the interior space.

In certain exemplary, non-limiting embodiments, the portable odor removal apparatus further includes a controller having an indicator capable of indicating when the battery requires maintenance.

In certain exemplary, non-limiting embodiments, the indicator is one or more of a visual indicator and a sonic indicator.

In certain exemplary, non-limiting embodiments, the battery is oriented horizontally.

In certain exemplary, non-limiting embodiments, the housing wall suggests the appearance of one or more of a fruit, a vegetable and a foodstuff

In certain exemplary, non-limiting embodiments, the exit passageway includes multiple exit passageways, at least one of which has a form suggesting the appearance of at least a portion of one or more of a fruit and a vegetable.

In certain exemplary, non-limiting embodiments, the housing wall includes a lower housing wall having a longitudinal surface curvature and an upper housing wall in mating relationship with the lower housing wall; the upper housing wall including a top surface and opposing first and sidewalls oriented generally perpendicular to the top surface.

### BRIEF DESCRIPTION OF THE DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following Figures:

FIG. 1 is a perspective view of an embodiment of an odor removal apparatus having improved odor removal and performance characteristics;

FIG. 2 is an exploded perspective view of the embodiment of FIG. 1;

FIG. 3A is a perspective view of one of the components of the embodiment of FIG. 2;

FIG. 3B is a partial cross section of the embodiment of FIG. 3A;

FIG. 4A is a perspective view of an alternative embodiment of a component of the embodiment of FIG. 2;

FIG. 4B is a partial cross section of the embodiment of FIG. 4A;

FIG. 5A is a perspective view of another alternative embodiment of a component of the embodiment of FIG. 2;

FIG. 5B is a partial cross section of the embodiment of FIG. 5A;

FIG. 6 is a cross sectional view along plane 6-6 of the embodiment of FIG. 1;

FIG. 7 is a perspective view of another embodiment of an odor removal apparatus having improved odor removal and performance characteristics; and

FIG. 8 is a perspective view of another embodiment of an odor removal apparatus of the present invention.

### DETAILED DESCRIPTION OF THE INVENTION

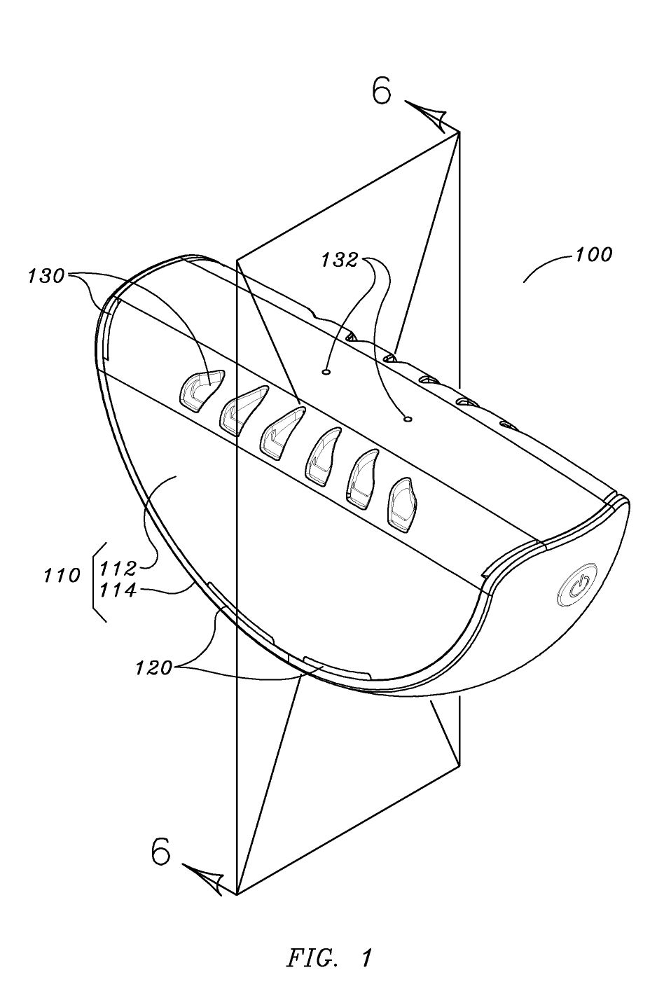

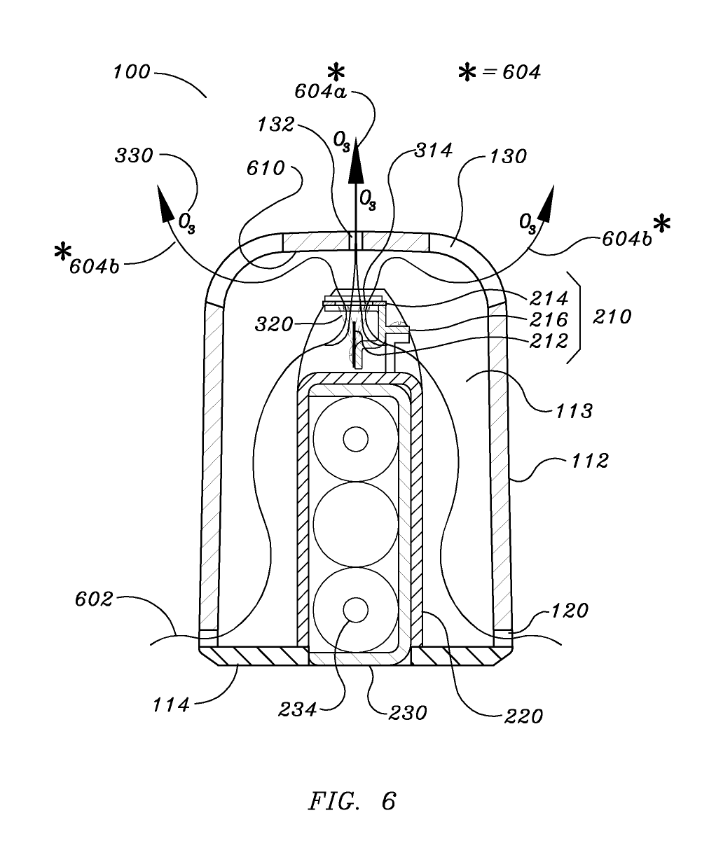

FIG. 1 is a front perspective view of an embodiment of the present invention of an odor removal apparatus 100 having improved odor removal and performance characteristics. Odor removal apparatus 100 includes housing wall 110, inlets 120, indirect exit passageways 130, and direct exit passageways 132. As shown, housing wall 110 includes upper housing wall 112 and lower housing wall 114. Also shown is section view 6-6 which corresponds to FIG. 6.

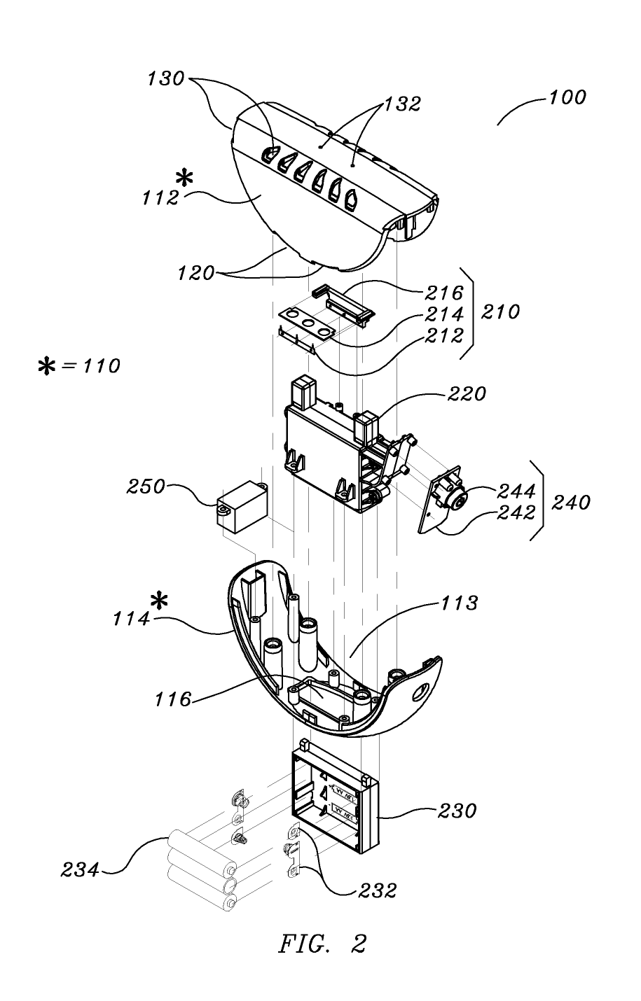

FIG. 2 is an exploded perspective view of the embodiment of FIG. 1. Housing 110 defines an interior space 113 in which the functional components of odor removal apparatus 100 are located. Ion generator 210 includes emitter electrode 212, attractor electrode 214 and may include mounting bracket 216. Mounting bracket 216 is used to maintain the position and location of attractor electrode 114 relative to emitter electrode 112. Controller 240 includes power control board 242 and user interface 244. Internal frame 220 is mounted to lower housing wall 114. Controller 240, Ion generator 210, and battery tray 230 are mounted to and/or located within interior space 113 by internal frame 220. Electrical connections (not shown) between the various components may be built within and or supported by internal frame 220. Capacitor 250 in the present embodiment is mounted to lower housing wall 114.

The power (voltage) source for odor removal apparatus 100 consists of at least one battery 234. Battery tray 230 may include battery contacts 232 located at opposing ends of multiple batteries 234. As shown batteries 234 are located within battery tray 230 and make electrical contact with battery contacts 232. When battery tray 232 and batteries 234 are subsequently inserted through battery access port 116 and inside internal frame 220 electrical contact is achieved between batteries 234 and controller 240, Ion generator 210 and capacitor 250.

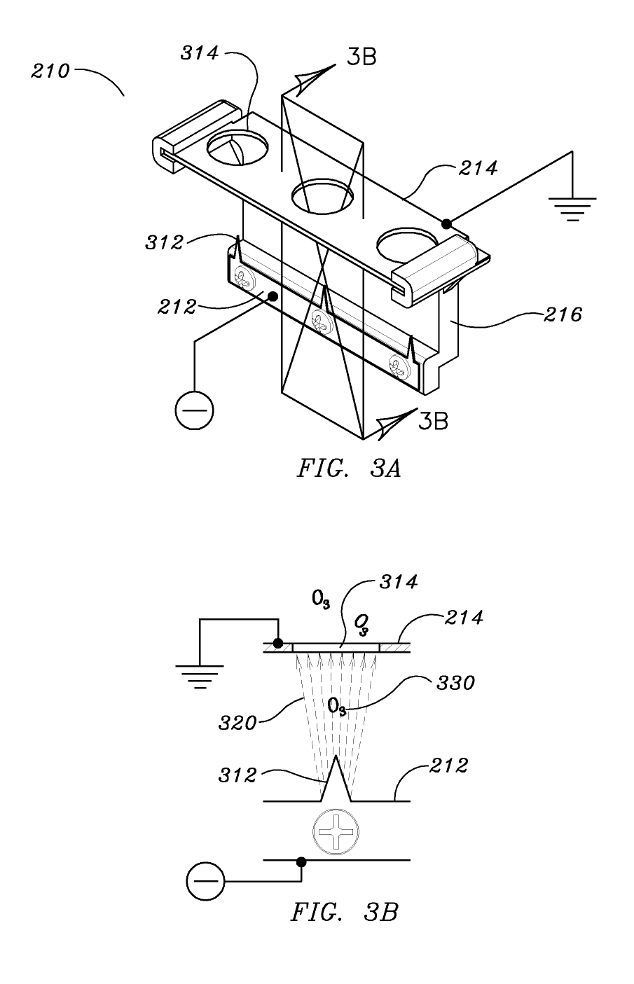

Controller 240 may be used to control one or more functions of odor removal apparatus 100. For example a first function may be the “on” and “off” status of the device. Power control board 242 may include timers or other devices to control the frequency and duration of power supplied to ion generator 210. It has been found that an intermittent power flow to ion generator 210 maintains an effective quantity of ozone 330 (see FIG. 3B) within an enclosed space (i.e. refrigerator) while simultaneously prolonging the useful life of batteries 234. For example ion generator 210 may be energized for an extended time period when odor removal apparatus 100 is first turned on and placed in the enclosed space. After the initial extended energized period, power flow to ion generator 210 may be interrupted initiating a prolonged non-energized period. After the prolonged non-energized period ion generator 210 may be energized for a brief energized period to renew the ozone 330 concentration level in the enclosed space. The cycle of alternating prolonged non-energized periods and brief energized periods greatly extends the useful life of batteries 234. For example it has been found that the initial extended energized period may be about 10 minutes followed by alternating prolonged non-energized periods of about 30 minutes and brief energized periods of between 1 to 2 minutes.

Controller 240 may also serve as an indicator regarding batteries 234 replacement needs. It is contemplated that the indicator may be a visual indicator, such as a light or a sonic device included on control board 242. For example, user interface 244 may be a lighted push button which illuminates for a fraction of one second every five or ten seconds. If batteries 234 require maintenance the cadence of the illumination of user interface 244 may change to illuminating for a fraction of one second every one or two seconds, thereby alerting the user that batteries 234 require maintenance.

FIG. 3A is a perspective view of ion generator 210 of FIG. 2. Ion generator frame 216 locates emitter electrode 212 and attractor electrode 214 in the proper location relative to one another. Also shown are ozone points 312 of emitter electrode 212 and multiple flow through passages 314 of attractor electrode 214. In the present example the electrical bias differential is created between negatively charged emitter electrode 212 and grounded attractor electrode 214. The total flow through area of ion generator 210 is defined by the combined area of flow through passages 314. Also shown is section plane 3B-3B.

FIG. 3B is a partial cross section along is section plane 3B-3B of FIG. 3A. When a voltage differential between emitter electrode 212 and attractor electrode 214 is induced ion field 320 is created as the ions travel from emitter electrode 212 toward attractor electrode 214. If the voltage differential is sufficient, oxygen in the ambient air will be converted to ozone 330.

As shown, emitter electrode 212 is negatively charged and attractor electrode 214 is grounded. As such negative ions are pulled from emitter electrode 212 creating ion field 320. It has been found that charging emitter electrode 212 with a negative charge increases the production of ozone 330 created by ozone points 312 when compared to charging emitter electrode 212 with a positive charge. It is contemplated that emitter electrode 212 could be positively or negatively charged while attractor electrode 214 could have the opposite charge in lieu of being grounded.

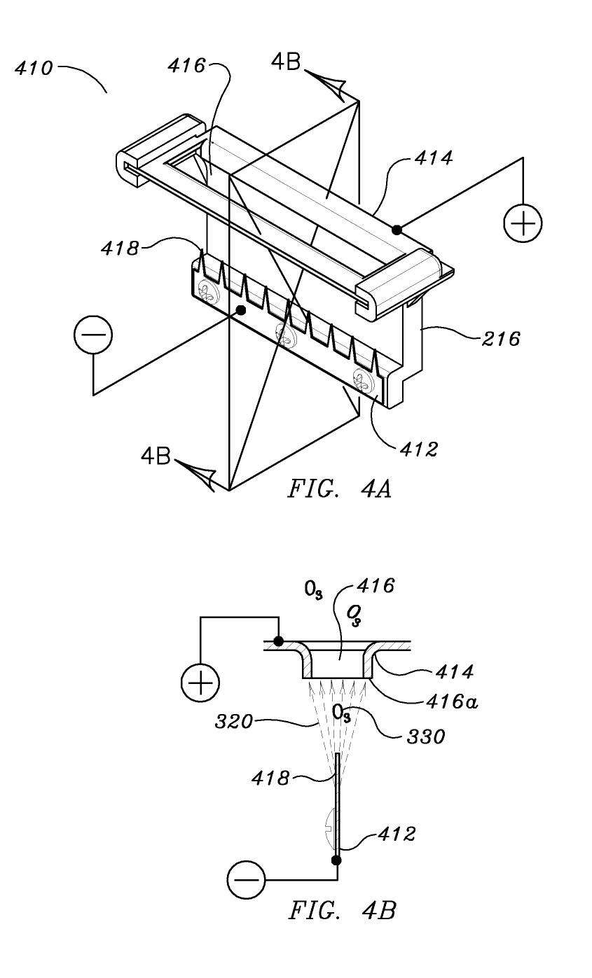

FIG. 4A is a perspective view the another embodiment of ion generator 410. Ion generator 410 includes ion generator frame 216 which locates emitter electrode 412 and attractor electrode 414 in the proper relative to one another. Also shown are ozone points 418 of emitter electrode 412 and flow through passage 416 of attractor electrode 414. The total flow through area of ion generator 410 is defined by the rectangular area of flow through passage 416. Also shown is section plane 4B-4B.

FIG. 4B is a partial cross section along is section plane 4B-4B of FIG. 4A. When a voltage differential between emitter electrode 412 and attractor electrode 414 is produced ion field 320 is created as the ions travel from emitter electrode 412 toward attractor electrode 414. If the voltage differential is sufficient, oxygen in the ambient air will be converted to ozone 330. As can be seen the passage edge 416a of attractor electrode 414 is located closer to multiple ozone points 418 when compared to the embodiment of FIG. 3B. This feature causes performance characteristic variations such as for example, the strength of ion field 320, quantity of ozone 330 created and other factors that can be changed to modify the performance of improved odor removal apparatus 100. In all other respects the embodiment of FIGS. 4A and 4B is similar to the embodiment of FIGS. 3A and 3B.

In the current embodiment emitter electrode 412 is negatively charged and attractor electrode 414 is positively charged. Using an opposite charge between emitter electrode 412 and attractor electrode 414 increases the voltage differential which increases the intensity of ion field 320 and the production of ozone 330. One disadvantage of using an opposite charge between emitter electrode 412 and attractor electrode 414 is an increased power consumption which may require frequent maintenance of batteries 234.

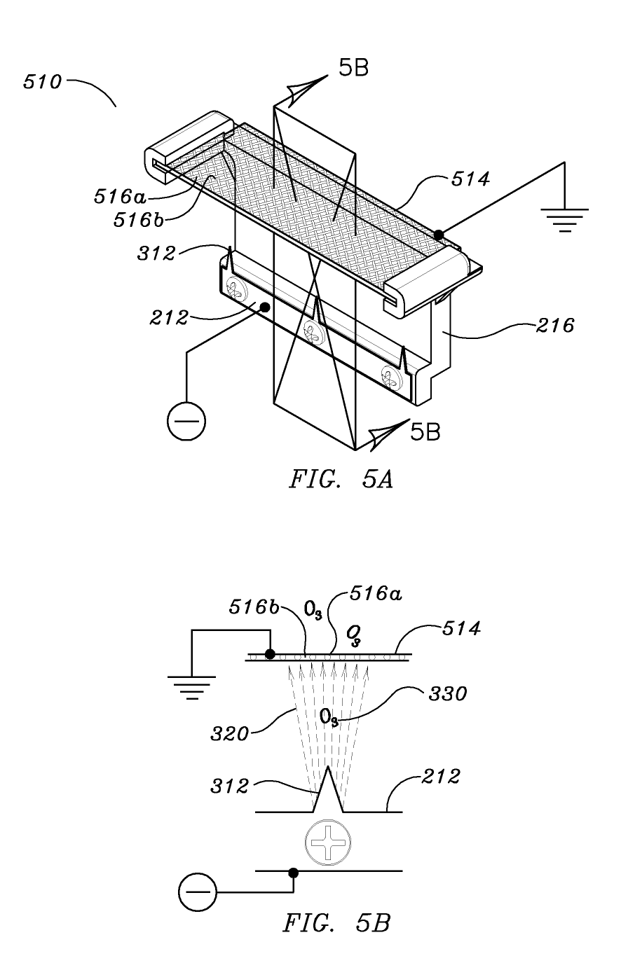

FIG. 5A is a perspective view the another embodiment of ion generator 510. Ion generator frame 216 locates emitter electrode 212 and attractor electrode 514 in the proper location relative to one another. As shown attractor electrode 514 is fabricated of electrically conductive mesh or screen, for example wire 516a which define flow through passages 516b there between. In the present example the electrical bias differential is created between negatively charged emitter electrode 212 and grounded attractor electrode 514. The total flow through area of ion generator 510 is defined by the combined area of flow through passages 516b. Also shown is section plane 5B-5B.

FIG. 5B is a partial cross section along is section plane 5B-5B of FIG. 5A. When a voltage differential between emitter electrode 212 and attractor electrode 514 is induced ion field 320 is created as the ions travel from emitter electrode 212 toward attractor electrode 514. If the voltage differential is sufficient, oxygen in the ambient air will be converted to ozone 330. In all other respects the embodiment of FIGS. SA and 5B is similar to the embodiment of FIGS. 3A and 3B.

As shown in FIGS. 3A, 4A and 5A ion generators 210, 410 and 510 have a total flow through areas associated with flow multiple through passages 314, 416 and 516b respectively. Although flow through passage(s) 314, 416 and 516b are shown as circular and rectangular the invention is not so limited. It is contemplated that other shapes, such as hexagons, triangles and other such polygon shapes might be used.

FIG. 6 is a cross sectional view along plane 6-6 of the embodiment of FIG. 1. As shown ion field 320 is created as the ions travel from emitter electrode 212 toward attractor electrode 214. The movement of the ions in ion field 320 entrains air molecules (not shown) and induces intake air 602 to enter interior space 113 defined by housing 112 through air inlets 120. Inlet air 602 travels through ion field 320, wherein inlet air 602 is mixed with ozone 330 (see FIG. 3B) as ozonized air 604. Ozonized air 604 exits odor removal apparatus 100 as direct flow 604a and indirect flow 604b.

As shown diffusion surface 610 (which may be non-conductive) is located directly above ion generators 210. Direct exits 132 are substantially in line with flow through passages 314 of attractor electrode 214. Indirect exits 130 are located to the side of flow through passages 314 of attractor electrode 214. The use of diffusion surface 610 in conjunction with direct exits 132 and indirect exits 130 disseminates ozone 330 more evenly within the enclosed space at the proper concentration as measured in parts per billion.

It has been found that to achieve proper dissemination and levels of ozone 330 in an enclosed space the total flow through area of direct exits 132 should be less than the total flow through area associated with flow through passages 314 while the total flow through area of indirect exits 130 should be equal to or greater than the total flow through area associated with flow through passages 314. The quantity of ozonized air 604 in the area proximate direct exits 132 is high when compared to locations proximate indirect exits 130. If the flow through area associated with direct exits 132 are too great the dissemination of ozonized air 604 throughout the enclosed space is less homogeneous because of excessive concentrations of ozone 330 in the area proximate direct exits 132.

Similarly the total flow through area of air inlets 120 is also central to the proper distribution of ozonized air 604. It has been found that the total flow through area of all air inlets 120 should be equal to or greater than the total flow through area associated with flow through passages 314.

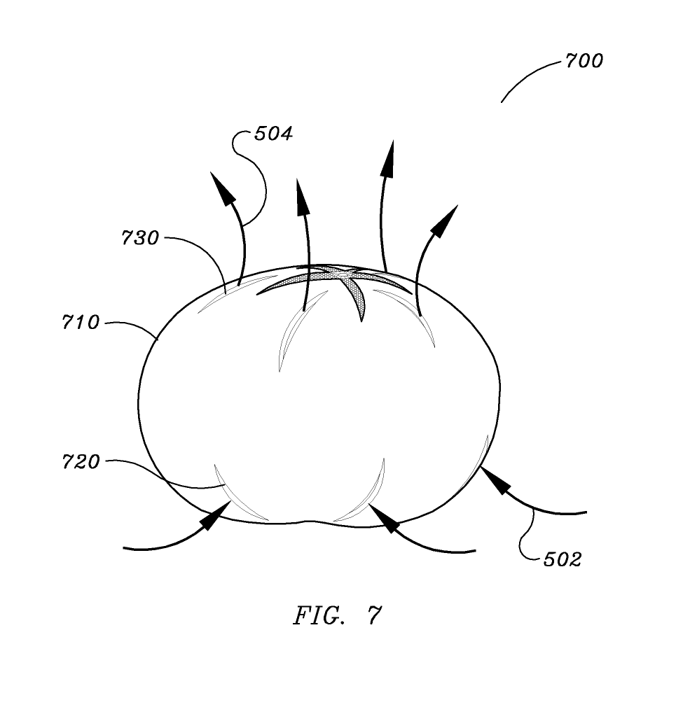

FIG. 7 is a perspective view of odor removal apparatus 700. Odor removal apparatus 700 includes housing wall 710, inlets 720, and exits 730. Inlet air 602 is drawn into housing wall 710 through inlets 720 and exits via exits 730 as ozonized air 604. As shown the overall form of odor removal apparatus 700 evokes the shape of a vegetable as opposed to the form of fruit evoked by odor removal apparatus 100 of FIG. 1. In all other respects odor removal apparatus 700 is similar to the embodiment of odor removal apparatus 100.

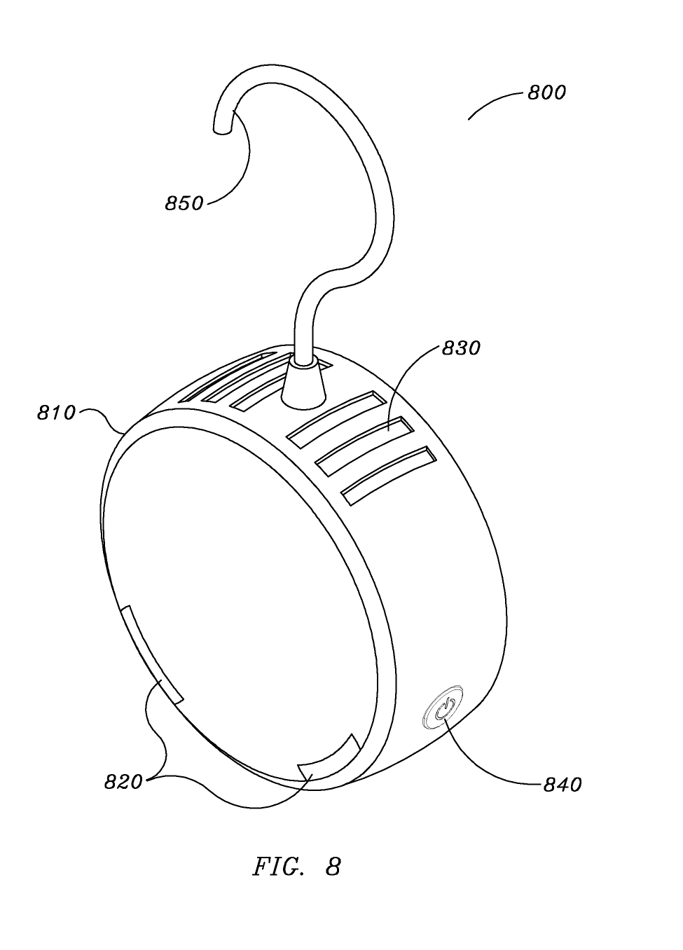

FIG. 8 is a perspective view of odor removal apparatus 800. Odor removal apparatus 800 includes housing wall 810, inlets 820, exits 830 controller 840, and hook 850. As shown odor removal apparatus 800 is designed to be hung in an enclosed space for example a closet. In all other respects odor removal apparatus 800 is similar to the embodiment of odor removal apparatus 100.

Once given the above disclosure, many other features, modifications, and improvements will become apparent to the skilled artisan. Such features, modifications, and improvements are therefore considered to be part of this invention, without limitation imposed by the example embodiments described herein. Moreover, any word, term, phrase, feature, example, embodiment, or part or combination thereof, as used to describe or exemplify embodiments herein, unless unequivocally set forth as expressly uniquely defined or otherwise unequivocally set forth as limiting, is not intended to impart a narrowing scope to the invention in contravention of the ordinary meaning of the claim terms by which the scope of the patent property rights shall otherwise be determined. All references discussed and disclosed herein are hereby incorporated by reference in their entirety.

What is claimed is:

1. A portable odor removal apparatus, comprising:
a housing wall defining an interior space;
an ion generator located in said interior space, said ion generator including an emitter electrode and an attractor electrode;
a voltage source; said voltage source capable of providing voltage to said ion generator;
an air inlet in said housing wall;
at least one non-conductive diffusion surface located above said ion generator; and
an exit passageway at least partially located in said non-conductive surface;
wherein, voltage provided to said ion generator from said voltage source induces an electrical bias differential between said emitter electrode and said attractor electrode; said electrical bias differential creating an ion field having a flow of ions from said emitter electrode toward said attractor electrode resulting in the production of ozone; and said flow of ions entraining air molecules to cause air outside said housing wall to enter said interior space through said air inlet and mix with said ozone to produce ozonized air; and
wherein said ozonized air exits said interior space through said exit passageway.

2. A portable odor removal apparatus according to claim 1, wherein said attractor electrode defines a flow through area.

3. A portable odor removal apparatus according to claim 2, wherein said exit passageway includes multiple direct exit passageways defining a total direct exit area; said total direct exit area being equal to or less than said flow through area defined by said attractor electrode.

4. A portable odor removal apparatus according to claim 2, wherein said exit passageway includes multiple indirect exit passageways defining a total indirect exit area; said total indirect exit area being equal to or greater than said flow through area defined by said attractor electrode.

5. A portable odor removal apparatus according to claim 2, wherein said air inlet includes multiple air inlets defining a total inlet area; said total inlet area being equal to or greater than said flow through area defined by said attractor electrode.

6. A portable odor removal apparatus according to claim 1, further comprising a controller including a timer capable of cycling said electrical bias differential on and off at predetermined time intervals.

7. A portable odor removal apparatus according to claim 1, wherein said electrical bias differential results from a negative charge on said emitter electrode and a grounding of said attractor electrode.

8. A portable odor removal apparatus according to claim 1, wherein said electrical bias differential results from a first charge having a first polarity on said emitter electrode and a second charge having a second polarity on said attractor electrode, wherein said first and said second polarities are opposite.

9. A portable odor removal apparatus according to claim 1, wherein said voltage source includes at least one battery located within said interior space.

10. A portable odor removal apparatus according to claim 9, further comprising a controller including an indicator capable of indicating when said at least one battery requires maintenance.

11. A portable odor removal apparatus according to claim 10, wherein said indicator is one or more of a visual indicator and a sonic indicator.

12. A portable odor removal apparatus according to claim 9, wherein said at least one battery is oriented horizontally.

13. A portable odor removal apparatus according to claim 1, wherein said housing wall suggests the appearance of one or more of a fruit, a vegetable and a foodstuff.

14. A portable odor removal apparatus according to claim 13, wherein said exit passageway includes multiple exit passageways; and wherein at least one of said multiple exit passageways has a form suggesting the appearance of at least a portion of one or more of a fruit and a vegetable.

15. A portable odor removal apparatus according to claim 13, wherein said housing wall includes a lower housing wall having a longitudinal surface curvature and an upper housing wall in mating relationship with said lower housing wall; said upper housing wall including a top surface and opposing first and sidewalls oriented generally perpendicular to said top surface.

16. A portable odor removal apparatus, comprising:
a housing wall defining an interior space;
an ion generator located in said interior space; said ion generator including an emitter electrode and an attractor electrode;
a voltage source located within said interior space and including at least one battery; said voltage source being capable of providing voltage to said ion generator;
an air inlet in said housing wall;
an exit passageway in said housing wall; and
a controller including a timer;
wherein, voltage provided to said ion generator from said voltage source induces an electrical bias differential between said emitter electrode and said attractor electrode; said electrical bias differential creating an ion field having a flow of ions from said emitter electrode toward said attractor electrode resulting in the production of ozone; and said flow of ions entraining air molecules to cause air outside said housing wall to enter said interior space through said air inlet and mix with said ozone to produce ozonized air;
wherein said ozonized air exits said interior space through said exit passageway; and
wherein said timer is capable of cycling said electrical bias differential on and off at predetermined time intervals.

17. A portable odor removal apparatus according to claim 16, wherein said controller further includes an indicator capable of indicating when said at least one battery requires maintenance.

18. A portable odor removal apparatus according to claim 17, wherein said indicator is one or more of a visual indicator and a sonic indicator.

19. A portable odor removal apparatus according to claim 16, wherein said at least one battery is oriented horizontally.

20. A portable odor removal apparatus according to claim 16, wherein said housing wall suggests the appearance of one or more of a fruit, a vegetable and a foodstuff.

21. A portable odor removal apparatus according to claim 20, wherein said exit passageway includes multiple exit passageways; and wherein at least one of said multiple exit passageways has a form suggesting the appearance of at least a portion of one or more of a fruit and a vegetable.

22. A portable odor removal apparatus according to claim 16, wherein said electrical bias differential results from a negative charge on said emitter electrode and a grounding of said attractor electrode.

23. A portable odor removal apparatus according to claim 16, wherein said electrical bias differential results from a first charge having a first polarity on said emitter electrode and a second charge having a second polarity on said attractor electrode; said first polarity and said second polarity being opposite.
